(19) United States
(12) Patent Application Publication
(10) Pub. No.: US 2026/0087591 A1
(43) Pub. Date: Mar. 26, 2026

(54) SYSTEM FOR IMPROVING VISIBILITY OF MEDICAL IMAGES, AND METHOD OF IMPROVING VISIBILITY USING THE SAME

(71) Applicant: CLARIPI INC., Seoul (KR)

(72) Inventors: Jong Hyo KIM, Seoul (KR); Chang Young Heo, Seoul (KR); Chul Kyun Ahn, Seoul (KR); Min Su Kim, Seoul (KR); Hyung Ki Kim, Seoul (KR)

(21) Appl. No.: 18/960,923

(22) Filed: Nov. 26, 2024

(30) Foreign Application Priority Data
Sep. 24, 2024 (KR) 10-2024-0129202

(51) Int. Cl. G06T 5/50; A61B 6/00; G06T 5/30; G06T 5/70; G06T 5/73; G06T 5/94; G06V 10/25; G06V 10/26

(52) U.S. Cl. CPC G06T 5/50; G06T 5/30; G06T 5/70; G06T 5/73; G06T 5/94; G06V 10/25; G06V 10/267; A61B 6/5258; G06T 2207/10081; G06T 2207/20081; G06T 2207/20221; G06T 2207/30061; G06T 2207/30096; G06V 2201/03

(57) ABSTRACT

Disclosed is a system for improving visibility of a lesion, the system including: a visibility v improvement device configured to implement a function of improving the visibility of the lesion contained in a medical image, the function including: segmenting an organ region of interest and a surrounding region by inputting the medical image to a deep learning model trained in advance, generating rendering images suitable for improving the visibility of the organ region of interest and the surrounding region, respectively, and generating a readable image by merging the rendering image of the organ region of interest and the rendering image of the surrounding region.

### CROSS REFERENCE TO RELATED APPLICATION

Priority to Korean Patent Application No. 10-2024-0129202 filed on Sep. 24, 2024, the entire disclosure of which is incorporated by reference herein, is claimed.

### BACKGROUND OF THE INVENTION

### Field of the Invention

The disclosure relates to a system for improving the visibility of lesions in medical images and a method of improving the visibility using the same, and more particularly to a system for improving the visibility of lesions in medical images, which improves the visibility of lesions in the organs of interest, and a method of improving the visibility using the same.

### Description of the Related Art

In the field of computed tomography (CT), maximum intensity projection (MIP) images are generally used to highlight high-density structures such as blood vessels, lungs, and bone tissue. In the MIP images, the strongest signal value among serval tomographic images is projected onto one image, thereby allowing complex anatomical structures or lesions to be easily identified. Thus, the MIP images are used in evaluation of high-density lesions such as vascular stenosis, lung lesions, and tumors.

In a conventional method of acquiring the MIP images, multi-layered tomographic images are taken using a computed tomography (CT) device, thereby acquiring images of several cross-sectional layers. When a desired anatomical structure or region of interest is selected, the strongest signal among pixels of each tomographic image within the selected region is projected to generate a two-dimensional MIP image. Then, the MIP images are reconstructed at various angles to assist in the diagnosis of the desired anatomical structure or region of interest.

However, the MIP image highlights only the high-density structures, and it is thus difficult to identify low-density lesions or surrounding structures, thereby making precise analysis and diagnosis difficult. In particular, because the high-density structures, such as muscles, fat, and the soft tissues around lungs are highlighted, it is difficult to identify lung lesions and the lung lesions adjacent to ribs are not clearly visible due to overlapping with the ribs.

### Documents of Related Art

(Patent Document) Korean Patent Publication No. 2021-0073047 (titled “METHOD FOR ARTIFICIAL INTELLIGENCE NODULE SEGMENTATION THROUGH MAXIMUM INTENSITY PROJECTION AND APPARATUS THEREOF,” and published on Jun. 18, 2021)

### SUMMARY OF THE INVENTION

An aspect of the disclosure is to provide a system for improving the visibility of lesions in medical images, in which the size and location of lesions (nodules, etc.) are more clearly identified in medical images such as a chest computed tomography (CT) images, and a method of improving the visibility using the same.

According to an embodiment of the disclosure, a system for improving visibility of a lesion includes: a visibility improvement device configured to implement a function of improving the visibility of the lesion contained in a medical image, the function including: segmenting an organ region of interest and a surrounding region by inputting the medical image to a deep learning model trained in advance, generating rendering images suitable for improving the visibility of the organ region of interest and the surrounding region, respectively, and generating a readable image by merging the rendering image of the organ region of interest and the rendering image of the surrounding region.

The rendering image may include at least one of a maximum intensity projection (MIP) image, a minimum intensity projection (MinIP) image, an average intensity projection (AIP) image, a volume rendering image, and a surface rendering image.

The segmentation of the organ region of interest and the surrounding region may include: performing segmentation based on a segmentation mask; and performing refinement and post-process for the segmented organ region of interest and the segmented surrounding region.

The refinement and post-process may include performing at least one of a morphology operation and a Gaussian blur technique.

After the refinement and post-process are completed, the function may include detecting the lesions from the organ region of interest and the surrounding region based on an automatic lesion detection algorithm.

After the detection of the lesions, the function may include defining regions of interest adjacent to the lesions in the organ region of interest and the surrounding region to adjust a dynamic size of the region of interest.

The function may include: setting a region of interest based on a distance between lesions upon multiple lesions, and setting the region of interest as one region upon the multiple lesions located within a preset distance, and setting the region of interest as individual regions upon a distance between the multiple lesions exceeding a preset distance.

After the refinement and post-process are completed, the function may include: generating multi-planar rendering images for the organ region of interest and multi-planar rendering images for the surrounding region, merging the multi-planar rendering images for the organ region of interest and the multi-planar rendering images for the surrounding region, and generating a readable image based on a combination of the merged image for the organ region of interest and the merged image for the surrounding region.

The multi-planar rendering images may include an axial image, a sagittal image and a coronal image.

The generation of the multi-planar rendering image may include adjusting contrast and brightness along a multi-planar direction.

The generation of the multi-planar rendering image may include removing noise and applying a contract enhancement technique from and to the multi-planar rendering image to highlight lesions.

The generation of the readable image may include transitioning or blending boundaries of the organ region of interest and the surrounding region contained in the readable image.

The blending may include applying an alpha blending technique to blend the boundaries, and generating a gradation between the boundaries.

The generation of the readable image may include adjusting at least one of contrast and sharpness to highlight the region.

The generation of the readable image may include highlighting the lesion in a different color.

The visibility improvement device may restore the projection image to the multi-planar rendering images.

Meanwhile, according to an embodiment of the disclosure, a method of improving visibility of a lesion contained in a medical image includes: segmenting an organ region of interest and a surrounding region by inputting the medical image to a deep learning model trained in advance, generating rendering images suitable for improving the visibility of the organ region of interest and the surrounding region, respectively, and generating a readable image by merging the rendering image of the organ region of interest and the rendering image of the surrounding region.

Meanwhile, according to an embodiment of the disclosure, a system for improving visibility of a lesion includes: a visibility improvement device configured to implement a function of improving the visibility of the lesion contained in a medical image, the function including: segmenting a lung region and a torso region by inputting the medical image to a deep learning model trained in advance, generating rendering images suitable for improving the visibility of the lung region and the torso region, respectively, and generating a readable image by merging the rendering image of the lung region and the rendering image of the torso region.

Meanwhile, according to an embodiment of the disclosure, a method of improving visibility of a lesion contained in a medical image includes: segmenting a lung region and a torso region by inputting the medical image to a deep learning model trained in advance, generating rendering images suitable for improving the visibility of the lung region and the torso region, respectively, and generating a readable image by merging the rendering image of the lung region and the rendering image of the torso region.

In the system for improving the visibility of medical images according to the disclosure, and the method of improving the visibility using the same method, the deep learning model is used to segment a specific organ of interest and surrounding tissues from the medical image so as to clearly identify the locations of the lesions and improve the sharpness of the lesions that are small or have unclear boundaries, thereby having an advantage of making it easier to identify relationships and locations between the lesions and the surrounding organs.

The technical effects of the disclosure are not limited to the aforementioned effects, and other unmentioned technical effects may become apparent to those skilled in the art from the following description.

### BRIEF DESCRIPTION OF THE DRAWINGS

FIG. 1 is a conceptual diagram showing a system for improving the visibility of medical images according to an embodiment,

FIG. 2 is a flowchart of a method of improving the visibility using a system for improving the visibility of medical images according to an embodiment, and

FIG. 3 is a conceptual diagram showing a method of improving the visibility using a system for improving the visibility of medical images according to an embodiment.

### DETAILED DESCRIPTION OF THE INVENTION

Hereinafter, embodiments of the disclosure will be described in detail with reference to the accompanying drawings. However, the disclosure is not limited to the embodiments set forth herein, but may be implemented in various different ways. The embodiments are provided to only complete the disclosure and allow those skilled in the art to understand the category of the disclosure. In the accompanying drawings, the shape, etc. of an element may be exaggerated for clear description, and like numerals refer to like elements.

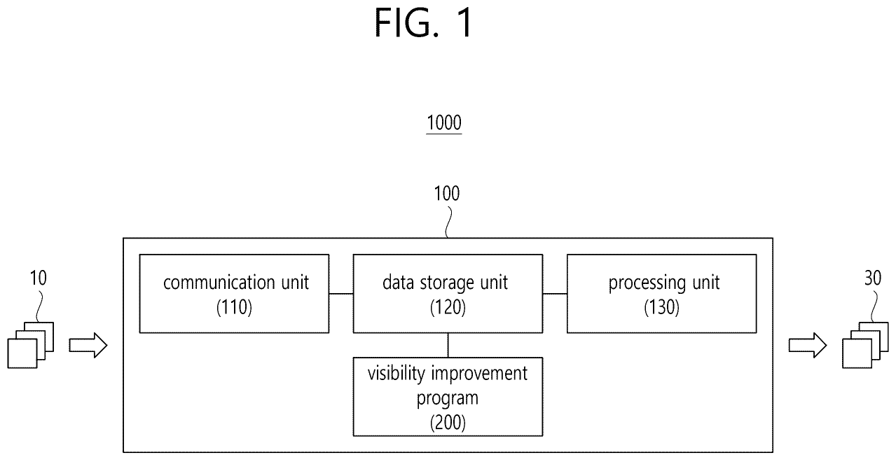

FIG. 1 is a conceptual diagram showing a system for improving the visibility of medical images according to an embodiment.

As shown in FIG. 1, a system 1000 for improving the visibility of medical images (hereinafter referred to as an improvement system includes a visibility improvement device 100 that recognizes a lesion from a medical image 10 acquired by a computed tomography device and generates a readable image 30 improved in the visibility of the lesion.

The visibility improvement device 100 is implemented by an electronic device in which a visibility improvement program 200 operating based on a deep learning model trained in advance can be installed, and may include various electronic devices with a display, such as a personal computer (PC), a netbook, a tablet PC, and a smartphone.

The visibility improvement device 100 includes a communication unit 110, a data storage unit 120, and a processing unit 130.

The communication unit 110 receives medical images 10 from a computed tomography (CT) device or a separate storage device, and transmits data to an external device as necessary. In addition, the data storage unit 120 includes a memory, and stores the visibility improvement program 200. The visibility improvement program 200 may perform an operation to improve the visibility of the medical image 10 and output an improved readable image 30 to medical staff. Further, the processing unit 130 may perform overall control for the visibility improvement device 100, and generate and execute a process for the operation to improve the visibility based on the visibility improvement program 200.

Below, a method of improving the visibility using the improvement system 1000 according to an embodiment will be described in detail with reference to the accompanying drawings.

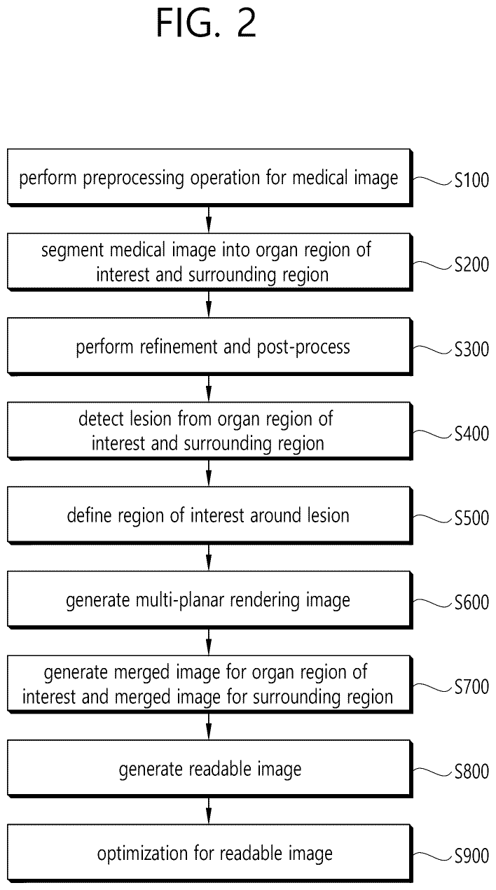

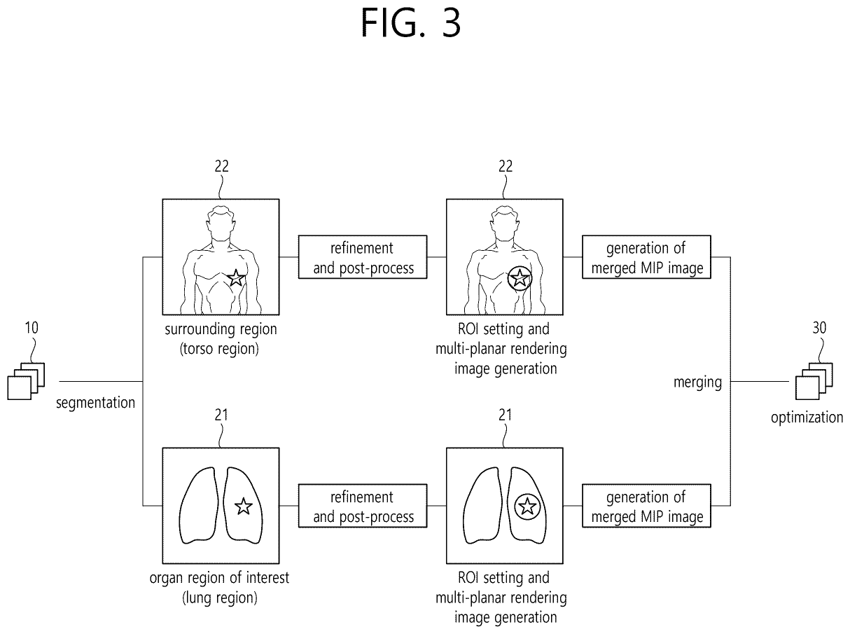

FIG. 2 is a flowchart of a method of improving the visibility using a system for improving the visibility of medical images according to an embodiment, and FIG. 3 is a flowchart of a method of improving the visibility using a system for improving the visibility of medical images according to an embodiment.

As shown in FIGS. 2 and 3, by the method of improving the visibility using the improvement system 1000 according to an embodiment, the medical image 10 is received through the communication unit 110. In addition, the processing unit 130 may generate and execute the following process based on the visibility improvement program 200.

First, the processing unit 130 may call the medical image 10 and perform a preprocessing operation for the medical image 10 (S100). The medical image 10 is given in a standard format of digital imaging and communication in medicine (DICOM), and includes a plurality of slides. Here, the preprocessing operation may refer to all operations for processing the medical image 10 suitable for subsequent analysis, such as data manipulation, data handling, and data cleaning.

Meanwhile, when the preprocessing operation for the medical image 10 is completed, the processing unit 130 segments the medical image 10 into a lung region 21 and a torso region 22 through the deep learning model trained in advance (S200). Here, the processing unit 130 uses a segmentation mask to define a boundary between the lung region 21 and the torso region 22. In addition, the processing unit 130 uses the deep learning model to automatically segment only the lung region 21 excluding the tissues of the heart and bones from the torso region 22.

Then, when the lung region 21 and the torso region 22 are segmented, the processing unit 130 uses the deep learning model trained in advance to refine and post-process each of the lung region 21 and the torso region 22 (S300). For example, in the refinement and post-process of the lung region 21 and the torso region 22, the morphology operation and the Gaussian blur technique may be applied in sequence.

The morphology operation employs erosion and dilation. The erosion reduces the boundary region of the mask to remove small noise or incorrect regions, and has an advantage of removing small defects within the mask. Further, the dilation compensates for defects remaining after the application of the erosion, and allows the boundary of the mask to smoothly expand. In addition, the Gaussian blur technique perform smoothing for the boundary of the mask. The Gaussian blur technique alleviates the sharp boundary of the mask by smoothing the boundary.

Although this embodiment describes that the processing unit 130 sequentially performs the morphology operation and the Gaussian blur technique, this is merely for describing this embodiment. Alternatively, the morphology operation and the Gaussian blur technique may be performed selectively.

Then, the processing unit 130 detects lesions from the lung region 21 and the torso region 22 based on an automatic lesion detection algorithm of the visibility improvement program 200 (S400). Here, the processing unit 130 not only automatically detects the lesions from the lung region 21 and the torso region 22 but also analyze the size, density and the like characteristics of the lesions.

Further, the processing unit 130 may accurately identify the location of the lesion by extracting the center coordinates and size information of the detected lesions. For example, the processing unit 130 sets a region of interest (ROI) including the lesions in the lung region 21 and the torso region 22. In addition, the processing unit 130 calculates the center coordinates of the lesions using the automatic lesion detection algorithm. Here, the automatic lesion detection algorithm may extract the size information about the ROI based on the calculated center coordinates of the lesion.

Then, the processing unit 130 defines the ROI around the lesion in the lung region 21 and the torso region 22 (S500). Here, the processing unit 130 defines the ROI around the lesions, and adjusts a dynamic size of the ROI according to the size of the lesion. The dynamic size adjustment may be applied to be concentrated only in a correct region corresponding to the size of the lesion. For example, in the case of a small lesion, the ROI may be concentrated in a small region, and in the case of a large lesion, the size of the ROI may be expanded.

Here, when there are multiple lesions, the processing unit 130 sets the ROI by taking a distance between the lesions into account. In other words, when multiple lesions exist in the lung region 21 and the torso region 22, the processing unit 130 segments the regions in consideration of the distances between the lesions, and sets the legions located close to each other as one ROI. On the other hand, when the distance between the lesions is long, the processing unit 130 may set the locations of multiple lesions as individual regions and process the lesions independently.

Then, when the ROI is set, the processing unit 130 generates a multi-planar rendering image for each of the lung region 21 and the torso region 22 (S600). For example, the processing unit 130 generates axial, sagittal and coronal multi-planar rendering images to extract ROIs from various planes of computed tomography images. Here, the multi-planar rendering images may include a maximum intensity projection (MIP) image, a minimum intensity projection (MinIP) image, an average intensity projection (AIP) image, a volume rendering image, a surface rendering image, etc.

In this case, the processing unit 130 adjusts adaptive window level and width in consideration of the characteristics of the corresponding plane. The window level and width refer to the standards for adjusting the brightness and the contrast in a computed tomography image, and the processing unit 130 applies the adaptive window level and width to adjust the contrast and brightness for each plane so that the lesions can be clearly visible. Further, the processing unit 130 removes noise from the multi-planar rendering image and applies contrast enhancement techniques, thereby ensuring that the lesions and surrounding tissue are clearly distinguished later.

Here, noise removal and contrast enhancement techniques may be achieved using the deep learning model trained in advance or through various image processing techniques.

Meanwhile, when the multi-planar rendering images for the lung region 21 and the torso region 22 are generated, the processing unit 130 merges the axial, sagittal and coronal multi-planar rendering images to generate one merged rendering image for the lung region 21 and one merged rendering image for the torso region 22 (S700). Here, the one merged rendering image may include an MIP image, a MinIP image, an AIP image, a volume rendering image, a surface rendering image, etc.

In addition, the processing unit 130 may perform rendering suitable for the characteristics of organs according to the types of the organs in the generation of the merged rendering images. For example, according to an embodiment, the lung region 21 includes a large air space, and thus the organ may be processed to become transparent. However, according to an alternative embodiment, when a liver or heart region is analyzed, the organ may be processed to become opaque suitably for the characteristics of the organ having high density.

Further, the processing unit 130 may generate the rendering image for the lung region 21 and the rendering image for the torso region 22 merged using the deep learning model trained in advance. However, this is merely for describing this embodiment, and there is no limit to a method of generating the rendering image.

Meanwhile, when the rendering image for the lung region 21 and the rendering image for the torso image 22 are completed to be improved in visibility from the conventional medical image 10, the processing unit 130 merges the rendering images, thereby generating the readable image 30 in which the boundaries between the lung region 21 and the torso region 22 are naturally combined (S800). Here, the processing unit 130 may define the boundary, i.e., an overlapping area between the lung region 21 and the torso region 22, or apply a gradual blending technique to realize a smooth image in the read image 30.

For example, the processing unit 130 may define an overlapping area between the boundaries of the lung region 21 and the torso region 22, so that the boundaries of the boundaries of the lung region 21 and the torso region 22 can be smoothly connected through mutual transitions of the regions adjacent to the boundaries. Further, the processing unit 130 may apply an alpha blending technique to smoothly blend the boundaries, thereby generating a natural image with a gradation effect between the boundaries.

Here, the processing unit 130 may generate a natural readable image 30 through the deep learning model trained in advance, but is not limited thereto.

Then, the processing unit 130 performs optimization for the read image 30 (S900). For example, the processing unit 130 may perform global contrast and sharpness adjustments, and perform selective color mapping to highlight the lesions.

In this case, the processing unit 130 applies the global contrast and sharpness adjustment to the readable image 30, so that the contrast between the lesions and the surrounding tissues can be optimized, thereby making the lesions clearly visible. In addition, the processing unit 130 may highlight the lesions in red to visually emphasize the lesions so that the lesions can be easily identified in the readable image 30.

Meanwhile, when the readable image 30 improved in visibility through the foregoing process is generated, the readable image 30 may be provided to medical staff through the visibility improvement program 200. In this case, the visibility improvement program 200 may provide various analysis functions. For example, the visibility improvement program 200 may reconstruct the readable image 30 into axial, sagittal and coronal multi-planar rendering images. In other words, the visibility improvement program 200 can restore the readable image 30 to the axial, sagittal and coronal multi-planar rendering images of when generating the multi-planar rendering images. Here, the visibility improvement program 200 may reconstruct the readable image 30 using the deep learning model trained in advance. For example, the deep learning model is trained based on the overall visibility improvement process including the generation of the multi-planar rendering image, thereby an having advantage of accurately and quickly restoring the axial, sagittal and coronal multi-planar rendering images from the readable image 30.

In this way, the visibility improvement program 200 may simultaneously generate the multi-planar rendering images from the readable image 30 in real time. Thus, the visibility improvement program 200 enables an efficient analysis of the location and characteristics of the lesions through cross-referencing functions between the planes. For example, the visibility improvement program 200 may simultaneously generate the multi-planar rendering images for the readable image 30 in the axial, sagittal and coronal planes, so that medical staff can analyze the shape and locations of the lesion from various angles when viewed from various angles. In addition, the visibility improvement program 200 allows the lesions identified in one plane to be easily confirmed in other planes, thereby having an advantage of effectively performing the analysis of the lesions.

Meanwhile, according to an embodiment, the lung region 21 and the torso region 22 are segmented from the medical image 10 containing the lesions, and the multi-plane images for each region are generated, thereby ultimately generating the readable image 30 in which the lung region 21 and the torso region 22 are recombined.

However, this is merely for describing this embodiment, and the improvement system 1000 may segment a region of another organ of interest (a lung region) and a surrounding region (a torso region), generate multi-plane images for each region, and ultimately generate the readable image 30 where the organ region of interest and the surrounding region are recombined.

Accordingly, in the system for improving the visibility of medical images according to the disclosure, and the method of improving the visibility using the same method, the deep learning model is used to segment a specific organ of interest and surrounding tissues from the medical image so as to clearly identify the locations of the lesions and improve the sharpness of the lesions that are small or have unclear boundaries, thereby having an advantage of making it easier to identify relationships and locations between the lesions and the surrounding organs.

Although a few embodiments of the disclosure have been described above and illustrated in the accompanying drawings, the embodiments should not be construed as limiting the technical spirit of the disclosure. The scope of the disclosure is limited only by the subject matters disclosed in the appended claims, and the technical spirit of the disclosure may be modified and changed in various forms by a person having ordinary knowledge in the art. Therefore, such modification and change obvious to those skilled in the art will fall within the scope of the disclosure.

What is claimed is:

1. A system for improving visibility of a lesion, the system comprising:
a visibility improvement device configured to implement a function of improving the visibility of the lesion contained in a medical image,
the function comprising:
segmenting an organ region of interest and a surrounding region by inputting the medical image to a deep learning model trained in advance,
generating rendering images suitable for improving the visibility of the organ region of interest and the surrounding region, respectively, and
generating a readable image by merging the rendering image of the organ region of interest and the rendering image of the surrounding region.

2. The system of claim 1, wherein the rendering image comprises at least one of a maximum intensity projection (MIP) image, a minimum intensity projection (MinIP) image, an average intensity projection (AIP) image, a volume rendering image, and a surface rendering image.

3. The system of claim 1, wherein the segmentation of the organ region of interest and the surrounding region comprises:
performing segmentation based on a segmentation mask; and
performing refinement and post-process for the segmented organ region of interest and the segmented surrounding region.

4. The system of claim 3, wherein the refinement and post-process comprises performing at least one of a morphology operation and a Gaussian blur technique.

5. The system of claim 3, wherein, after the refinement and post-process are completed,
the function comprises detecting the lesions from the organ region of interest and the surrounding region based on an automatic lesion detection algorithm.

6. The system of claim 5, wherein, after the detection of the lesions,
the function comprises defining regions of interest adjacent to the lesions in the organ region of interest and the surrounding region to adjust a dynamic size of the region of interest.

7. The system of claim 6, wherein the function comprises:
setting a region of interest based on a distance between lesions upon multiple lesions, and
setting the region of interest as one region upon the multiple lesions located within a preset distance, and setting the region of interest as individual regions upon a distance between the multiple lesions exceeding a preset distance.

8. The system of claim 3, wherein, after the refinement and post-process are completed,
the function comprises:
generating multi-planar rendering images for the organ region of interest and multi-planar rendering images for the surrounding region,
merging the multi-planar rendering images for the organ region of interest and the multi-planar rendering images for the surrounding region, and
generating a readable image based on a combination of the merged image for the organ region of interest and the merged image for the surrounding region.

9. The system of claim 8, wherein the multi-planar rendering images comprises an axial image, a sagittal image and a coronal image.

10. The system of claim 9, wherein the generation of the multi-planar rendering image comprises adjusting contrast and brightness along a multi-planar direction.

11. The system of claim 9, wherein the generation of the multi-planar rendering image comprises removing noise and applying a contract enhancement technique from and to the multi-planar rendering image to highlight lesions.

12. The system of claim 8, wherein the generation of the readable image comprises transitioning or blending boundaries of the organ region of interest and the surrounding region contained in the readable image.

13. The system of claim 12, wherein the blending comprises applying an alpha blending technique to blend the boundaries, and generating a gradation between the boundaries.

14. The system of claim 8, wherein the generation of the readable image comprises adjusting at least one of contrast and sharpness to highlight the region.

15. The system of claim 8, wherein the generation of the readable image comprises highlighting the lesion in a different color.

16. The system of claim 9, wherein the visibility improvement device restores the projection image to the multi-planar rendering images.

17. A method of improving visibility of a lesion contained in a medical image, the method comprising:
segmenting an organ region of interest and a surrounding region by inputting the medical image to a deep learning model trained in advance,
generating rendering images suitable for improving the visibility of the organ region of interest and the surrounding region, respectively, and
generating a readable image by merging the rendering image of the organ region of interest and the rendering image of the surrounding region.

18. A system for improving visibility of a lesion, the system comprising:
a visibility improvement device configured to implement a function of improving the visibility of the lesion contained in a medical image,
the function comprising:
segmenting a lung region and a torso region by inputting the medical image to a deep learning model trained in advance,
generating rendering images suitable for improving the visibility of the lung region and the torso region, respectively, and
generating a readable image by merging the rendering image of the lung region and the rendering image of the torso region.

19. A method of improving visibility of a lesion contained in a medical image, the method comprising:
segmenting a lung region and a torso region by inputting the medical image to a deep learning model trained in advance,
generating rendering images suitable for improving the visibility of the lung region and the torso region, respectively, and
generating a readable image by merging the rendering image of the lung region and the rendering image of the torso region.
